(19) United States
(12) Patent Application Publication
(10) Pub. No.: US 2024/0226878 A1
(43) Pub. Date: Jul. 11, 2024

(54) OVERFILL-TOLERANT MICROFLUIDIC STRUCTURES

(71) Applicant: Hewlett-Packard Development Company, L.P., Spring, TX (US)

(72) Inventors: Michael W. Cumbie, Corvallis, OR (US); Viktor Shkolnikov, Palo Alto, CA (US); Brian J. Keefe, San Diego, CA (US)

(73) Assignee: Hewlett-Packard Development Company, L.P., Spring, TX (US)

(21) Appl. No.: 18/559,496

(22) PCT Filed: May 7, 2021
(86) PCT No.: PCT/US2021/031344

§ 371 Date: Nov. 7, 2023

(51) Int. Cl. B01L 3/00; B01L 7/00

(52) U.S. Cl. CPC B01L 3/502707; B01L 7/52; B01L 2200/0684; B01L 2300/048; B01L 2300/0838

(57) ABSTRACT

An example overfill-tolerant microfluidic structure can include an inlet microfluidic channel. A sample chamber can be connected to the inlet microfluidic channel to receive liquid from the inlet microfluidic channel. A gas-permeable liquid barrier can be connected to the sample chamber and positioned to allow gas to flow out of the sample chamber. An overflow chamber can be connected to the inlet microfluidic channel. A capillary break can be positioned between the inlet microfluidic channel and the overflow chamber. The capillary break can include a narrowed opening with a smaller width than a width of the inlet microfluidic channel. In some examples, the gas-permeable liquid barrier can allow gas to flow out of the sample chamber at a pressure lower than the break pressure, and prevent liquid from flowing out of the sample chamber at the break pressure.

### BACKGROUND

Microfluidics relates to the behavior, precise control and manipulation of fluids that are geometrically constrained to a small, typically sub-millimeter, scale. Numerous applications employ passive fluid control techniques such as capillary forces. In some applications, external actuation techniques are employed for a directed transport of fluid. A variety of applications for microfluidics exist, with various applications involving differing controls over fluid flow, mixing, temperature, evaporation, and so on.

### BRIEF DESCRIPTION OF THE DRAWINGS

Additional features of the disclosure will be apparent from the detailed description which follows, taken in conjunction with the accompanying drawings, which together illustrate, by way of example, features of the present technology.

FIGS. 1A-1D are schematic views of an example overfill-tolerant microfluidic structure in accordance with the present disclosure;

FIG. 2 is a schematic view of another example overfill-tolerant microfluidic structure in accordance with the present disclosure;

FIG. 3 is a schematic view of yet another example overfill-tolerant microfluidic structure in accordance with the present disclosure;

FIG. 4 is a schematic view of a different example overfill-tolerant microfluidic structure in accordance with the present disclosure;

FIG. 5 is a schematic view of another example overfill-tolerant microfluidic structure in accordance with the present disclosure;

FIG. 6 is a schematic view of still another example overfill-tolerant microfluidic structure in accordance with the present disclosure;

FIG. 7 is a schematic view of yet another example overfill-tolerant microfluidic structure in accordance with the present disclosure;

FIGS. 8A-8E are schematic views of another example overfill-tolerant microfluidic structure in accordance with the present disclosure;

FIGS. 9A-9F show various views of an example microfluidic device in accordance with the present disclosure; and

FIG. 10 is a flowchart illustrating an example method of heating a liquid sample in accordance with the present disclosure.

Reference will now be made to several examples that are illustrated herein, and specific language will be used herein to describe the same. It will nevertheless be understood that no limitation of the scope of the disclosure is thereby intended.

### DETAILED DESCRIPTION

The present disclosure describes overfill-tolerant microfluidic structures. In one example, an overfill-tolerant microfluidic structure includes an inlet microfluidic channel and a sample chamber connected to the inlet microfluidic channel to receive liquid from the inlet microfluidic channel. A gas-permeable liquid barrier is connected to the sample chamber and positioned to allow gas to flow out of the sample chamber. An overflow chamber is connected to the inlet microfluidic channel. A capillary break is positioned between the inlet microfluidic channel and the overflow chamber. The capillary break includes a narrowed opening with a smaller width than a width of the inlet microfluidic channel. In some examples, the capillary break can prevent liquid from passing up to a break pressure, where the gas-permeable liquid barrier allows gas to flow out of the sample chamber at a pressure lower than the break pressure, but prevents liquid from flowing out of the sample chamber at the break pressure. In certain examples, the overflow chamber can be connected upstream of the sample chamber. In some examples, the gas-permeable liquid barrier can include a gas-permeable liquid-impermeable membrane, a pore having a hydrophobic surface, a labyrinth seal, a dry hydrogel precursor, or a second capillary break having a smaller width than the capillary break between the inlet microfluidic channel and the overflow chamber. In further examples, the narrowed opening can have a width from 2 μm to 20 μm. The overfill-tolerant microfluidic structure can also include a second gas-permeable liquid barrier connected to the overflow chamber and positioned to allow gas to flow out of the overflow chamber. For example, gas in the overflow chamber can be displaced by liquid flowing into the overflow chamber from the inlet microfluidic channel. In still further examples, the sample chamber can include a first fraction chamber, a second fraction chamber upstream of the first fraction chamber, and a microfluidic connection channel connecting the first fraction chamber to the second fraction chamber. In another example, the overfill-tolerant microfluidic structure can also include a containment chamber connected downstream of the gas-permeable liquid barrier to contain aerosolized material that passes through the gas-permeable liquid barrier. In other examples, the sample chamber can include a bubble-excluding region having an area of increased hydrophilicity on an interior surface of the sample chamber compared to surrounding areas of the interior surface. In certain examples, the inlet microfluidic channel can include an in-line mixer. In further examples, the overfill-tolerant microfluidic structure can also include a bubble remover on the inlet microfluidic channel to remove gas bubbles from the liquid before the liquid flows into the sample chamber.

The present disclosure also describes microfluidic devices. In one example, a microfluidic device includes a substrate, a heater on or embedded in the substrate, and a microfluidic structure on the substrate. The microfluidic structure includes: an inlet microfluidic channel; a sample chamber connected to the inlet microfluidic channel to receive liquid from the inlet microfluidic channel, wherein the sample chamber is proximate to the heater; and a gas-permeable liquid barrier connected to the sample chamber and positioned to allow gas to flow out of the sample chamber. For example, gas in the sample chamber can be displaced by liquid flowing into the sample chamber from the inlet microfluidic channel. The device also includes an overflow chamber connected to the inlet microfluidic channel. In some examples, the overflow chamber can be connected upstream of the sample chamber. A capillary break is positioned between the inlet microfluidic channel and the overflow chamber. The capillary break includes a narrowed opening with a smaller width than a width of the inlet microfluidic channel. In some examples, the capillary break can prevent liquid from passing up to a break pressure. The gas-permeable liquid barrier can allow gas to flow out of the sample chamber at a pressure lower than the break pressure, but can prevent liquid from flowing out of the sample chamber at the break pressure. In some examples, the substrate can include glass, silicon, a printed circuit board, a polyimide film, plastic, metal, sapphire, or a combination thereof. In further examples, the gas-permeable liquid barrier can include a gas-permeable liquid-impermeable membrane, a pore having a hydrophobic surface, a labyrinth seal, a dry hydrogel precursor, or a second capillary break having a smaller width than the capillary break between the inlet microfluidic channel and the overflow chamber.

The present disclosure also describes microfluidic systems. In one example, a microfluidic system includes a sample cartridge that includes a substrate and a microfluidic structure on the substrate. The microfluidic structure includes: an inlet microfluidic channel; a sample chamber connected to the inlet microfluidic channel to receive liquid from the inlet microfluidic channel; a gas-permeable liquid barrier connected to the sample chamber and positioned to allow gas to flow out of the sample chamber as the gas is displaced by liquid flowing into the sample chamber from the inlet microfluidic channel; an overflow chamber connected to the inlet microfluidic channel upstream of the sample chamber; and a capillary break positioned between the inlet microfluidic channel and the overflow chamber. The capillary break includes a narrowed opening with a smaller width than a width of the inlet microfluidic channel. The capillary break prevents liquid from passing up to a break pressure. The gas-permeable liquid barrier allows gas to flow out of the sample chamber at a pressure lower than the break pressure, but prevents liquid from flowing out of the sample chamber at the break pressure. The system also includes a heater positioned proximate to the sample chamber, and a sensor positioned to measure a property of material in the sample chamber. In some examples, the heater can be on or embedded in the substrate of the sample cartridge. In further examples, the sample chamber can include a bubble-excluding region having an area of increased hydrophilicity on an interior surface of the sample chamber compared to surrounding areas of the interior surface, and the sensor can be positioned to measure a property of material in the bubble-excluding region.

Methods of heating a liquid sample are also described. In one example, a method of heating a liquid sample includes introducing a liquid into an inlet microfluidic channel. The liquid flows through the inlet microfluidic channel into a sample chamber that is connected to the inlet microfluidic channel. A gas permeable liquid barrier is connected to the sample chamber and positioned to allow gas to flow out of the sample chamber as the gas is displaced by the liquid flowing into the sample chamber from the inlet microfluidic channel. The liquid continues to flow into the sample chamber until the liquid contacts the gas permeable liquid barrier. After the liquid contacts the gas permeable liquid barrier, additional liquid flows through the inlet microfluidic channel into an overflow chamber that is connected to the inlet microfluidic channel. In some examples, the overflow chamber can be connected upstream of the sample chamber. A capillary break is positioned between the inlet microfluidic channel and the overflow chamber. The capillary break includes a narrowed opening with a smaller width than a width of the inlet microfluidic channel. The liquid in the sample chamber is then heated. In some examples, the liquid can include a target nucleic acid and a master mix reagent to amplify the target nucleic acid, and the heating can be repeated such that the target nucleic acid is amplified via a polymerase chain reaction process. In further examples, the method can include mixing the liquid using an in-line mixer in the inlet microfluidic channel upstream of the sample chamber.

It is noted that when discussing overfill-tolerant microfluidic structures, microfluidic devices, microfluidic systems, and methods, these discussions can be considered applicable to other examples whether or not they are explicitly discussed in the context of that example unless expressly indicated otherwise. Thus, for example, when discussing a chamber in an overfill-tolerant microfluidic structure, such disclosure is also relevant to and directly supported in context of microfluidic devices, systems, methods, and vice versa. Furthermore, for simplicity and illustrative purposes, the present disclosure is described by referring mainly to certain examples. In the following description, numerous specific details are set forth in order to provide a thorough understanding of the present disclosure. It will be readily apparent however, that the present disclosure can be practiced without limitation to some of these specific details. In other instances, certain methods, compounds, compositions, and structures have not been described in detail so as not to unnecessarily obscure the present disclosure.

### Overfill-Tolerant Microfluidic Structures

The present disclosure describes microfluidic structures that include a sample chamber that can be fillable with a liquid sample of interest. In various examples, the liquid that fills the chamber can be “of interest” because a property of the liquid can be measured, or because the liquid can undergo a particular chemical reaction, or for some other purpose. The microfluidic structure can also be overfill-tolerant, meaning that a liquid sample having a volume larger than the volume of the sample chamber can be introduced into the microfluidic structure. The microfluidic structure can be designed in a way such that the sample chamber can be filled with the liquid, and then when the sample chamber is full, excess liquid can be redirected to an overflow chamber.

In particular, in some examples, the overfill-tolerant microfluidic structure can include an inlet microfluidic channel that is connected to a sample chamber and an overflow chamber. The overflow chamber can be connected to the inlet microfluidic channel upstream of the sample chamber. A capillary break can be positioned between the inlet microfluidic channel and the overflow chamber. The capillary break can include a narrowed opening having a smaller width than the width of the inlet microfluidic channel. Surface tension can prevent liquid from passing through the capillary break until the liquid reaches a sufficient pressure to overcome the surface tension forces. This pressure can be referred to as the “break pressure” of the capillary break. Therefore, liquid can be introduced through the inlet microfluidic channel and the liquid can bypass the overflow chamber because the capillary break can prevent the liquid from flowing into the overflow chamber. The liquid can instead flow into the sample chamber and fill the sample chamber before the overflow chamber. The microfluidic structure can also include a gas-permeable liquid barrier connected to the sample chamber. As liquid flows into the sample chamber, the liquid can displace gas in the sample chamber and the gas can flow out through the gas-permeable liquid barrier. However, after the liquid has filled the sample chamber, the gas-permeable liquid barrier can prevent the liquid from leaving the sample chamber. The pressure of the liquid can then increase to overcome the break pressure of the capillary break, and excess liquid can flow into the overflow chamber. In this way, the sample chamber can be filled with a precise volume of liquid, and excess liquid can be contained in the overflow chamber.

Certain processes can involve samples of liquid that may have an unknown or variable volume. For example, some test procedures can involve a human loading a liquid sample into a microfluidic device by a syringe or pipette. The liquid sample itself can have some variability in volume, and the human worker may be imprecise in loading the sample into the microfluidic device, which can introduce additional variability. It can be difficult to design a microfluidic device that can contain and process such unknown or variable liquid sample volumes. The overfill-tolerant microfluidic structures described herein can be useful in such processes. In some examples, the liquid sample may have a known minimum volume, but the actual volume of liquid samples can be greater than the minimum volume, such as by 50% to 100% or more. An overfill-tolerant microfluidic system can be designed to have a sample chamber with a volume that is less than the minimum liquid sample volume. The overflow chamber can be designed to have a volume that is more than enough to contain the remainder of the liquid sample. Thus, even though the volume of actual liquid samples may vary, the sample chamber can reliably be filled with liquid and the overflow chamber can be partially filled with any excess liquid from the liquid sample.

In some particular examples, the overfill-tolerant microfluidic structure can be a part of a microfluidic device used for a process that involves heating of the sample liquid, such as the thermal cycling employed during polymerase chain reaction (PCR) nucleic acid amplification. In such examples, the sample chamber can be heated to increase the temperature of liquid in the sample chamber. Heating the sample liquid can cause some amount of thermal expansion of the liquid. Additionally, it is possible for air bubbles to be present in the sample chamber for a variety of reasons. Air bubbles can expand much more when heated compared to the thermal expansion of most liquids. The overfill-tolerant microfluidic structures described herein can easily accommodate such expansion because liquid can freely flow from the sample chamber into the partially-filled overflow chamber. If multiple thermal cycles are performed, then liquid can flow back and forth from the sample chamber to the overflow chamber and back again. This can allow the liquid in the sample chamber to be heated without creating unduly high pressure in the sample chamber that could potentially damage the microfluidic device.

A variety of additional useful features can be incorporated into the microfluidic structures described herein. In some examples, these features can include additional inlet channels, sample chambers that are divided into multiple smaller chambers, containment chambers for containing aerosolized materials, in-line mixers, bubble removers, bubble-excluding regions, and others. These examples are described in more detail below.

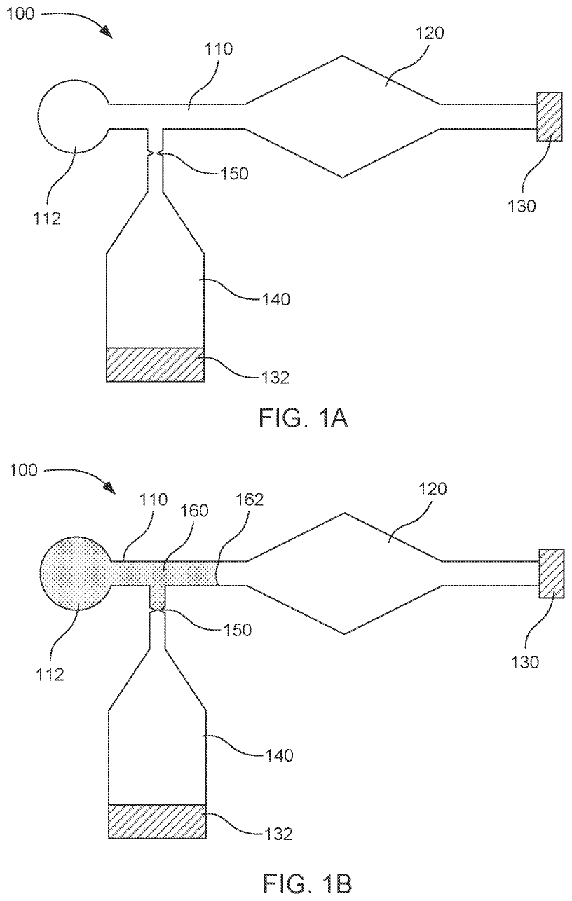

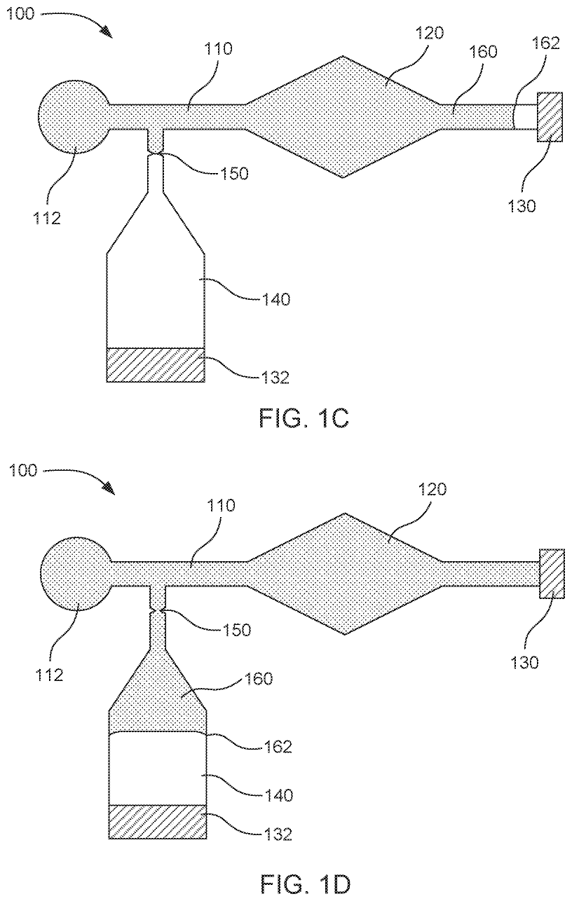

FIG. 1A shows a schematic view of one example overfill-tolerant microfluidic structure 100. The structure includes an inlet microfluidic channel 110. An inlet port 112 allows liquid to be introduced into the inlet microfluidic channel. A sample chamber 120 is connected to the inlet microfluidic channel to receive liquid from the inlet microfluidic channel. A gas-permeable liquid barrier 130 is connected to the sample chamber. The gas-permeable liquid barrier is positioned to allow gas to flow out of the sample chamber as the gas is displaced by liquid flowing into the sample chamber from the inlet microfluidic channel. An overflow chamber 140 is connected to the inlet microfluidic channel upstream of the sample chamber. A capillary break 150 is positioned between the inlet microfluidic channel and the overflow channel. The capillary break includes a narrowed opening that has a smaller width than the inlet microfluidic channel. The capillary break can prevent liquid from passing up to a break pressure. The gas-permeable liquid barrier can allow gas to flow out of the sample chamber at a pressure lower than the break pressure, but can prevent liquid from flowing out of the sample chamber at the break pressure. In this example, the overflow chamber also includes a second gas-permeable liquid barrier 132 to allow gas to exit the overflow chamber when liquid flows into the overflow chamber while also ensuring that the liquid does not escape from the overflow chamber. This figure shows the microfluidic structure before any liquid is introduced into the inlet microfluidic channel. In some examples, the microfluidic structure can be a part of a microfluidic device that is packaged in a dry state, without liquid in the inlet microfluidic channel or the chambers of the microfluidic structure. A sample liquid can later be introduced into the inlet microfluidic channel by a user.

FIG. 1B shows the example overfill-tolerant microfluidic structure 100 as liquid 160 begins to fill the inlet microfluidic channel 110. A sufficient pressure can be applied to the liquid to cause the liquid to flow through the inlet microfluidic channel. However, the pressure can be insufficient to overcome the break pressure of the capillary break 150. Therefore, the liquid does not flow into the overflow chamber 140. Instead, the liquid bypasses the overflow chamber and flows to the sample chamber 120. The liquid forms a meniscus 162 that moves along the inlet microfluidic channel.

FIG. 1C shows the same example overfill-tolerant microfluidic structure 100 after the liquid 160 has flowed into the sample chamber 120. In this figure, the meniscus 162 is approaching the gas permeable liquid barrier 130 that is downstream of the sample chamber. In this particular example, a segment of microfluidic channel is downstream of the sample chamber, and the segment of microfluidic channel leads to the gas permeable liquid barrier. However, in other examples, the gas permeable liquid barrier can be positioned directly on the sample chamber, such as at the downstream end of the sample chamber. At this point, the liquid still has not passed through the capillary break 150 because the liquid pressure is not sufficient to overcome the force of surface tension in the narrow opening of the capillary break.

FIG. 1D shows the example overfill-tolerant microfluidic structure 100 after the liquid 160 has reached the gas permeable liquid barrier 130 downstream of the sample chamber 120. The gas permeable liquid barrier prevents the liquid from flowing out of the sample chamber, and thus the pressure of the liquid begins to increase when the liquid becomes constrained in the sample chamber. The pressure of the liquid quickly reaches the break pressure of the capillary break 150. This allows the liquid to pass through the capillary break and start flowing into the overflow chamber 140. Once the liquid has passed through the capillary break, there is no longer a meniscus present in the narrow opening of the capillary break. Therefore, the surface tension forces that had previously prevented liquid from flowing through the capillary break are no longer present. At this point, liquid can easily flow into the overflow chamber at a lower pressure. In some examples, the liquid can partially fill the overflow chamber. As the liquid flows into the overflow chamber, the liquid displaces air that was in the overflow chamber. The air can flow out of the overflow chamber through the second gas permeable liquid barrier 132 that is positioned at the end of the overflow chamber.

In various examples, a gas permeable liquid barrier can be positioned to allow gas to escape from the sample chamber when liquid flows into the sample chamber from the inlet microfluidic channel. The gas that is displaced can be whatever gas is inside the sample chamber prior to introducing the liquid. In many cases, the gas can be air. However, in some cases the microfluidic structure may be packed in another gas, such as nitrogen, argon, carbon dioxide, or another gas. In some examples, the gas permeable liquid barrier can be positioned at an end of the sample chamber opposite from the inlet microfluidic channel. In this arrangement, liquid can flow into the sample chamber from the inlet microfluidic channel and the liquid can displace substantially all the gas and completely fill the sample chamber before the liquid contacts the gas permeable liquid barrier. In further examples, the microfluidic structure can include a microfluidic channel segment downstream of the sample chamber, and the microfluidic channel segment can lead from the sample chamber to the gas permeable liquid barrier.

The gas permeable liquid barrier can be a material or structure that can allow gas to pass through while preventing liquid from passing through. In some examples, liquid may be able to pass through the barrier when under a substantial pressure, but the barrier can prevent liquid from passing through up to or above the break pressure of the capillary break mentioned above. In some examples, the gas-permeable liquid barrier can be a gas-permeable liquid-impermeable membrane. Such membranes can include polymers such as polytetrafluoroethylene, polyethylene, polypropylene, polyvinylfluoride, polyvinylidene fluoride, fluorinated ethylene-propylene copolymer, or combinations thereof. Other examples can include porous membranes that are functionalized with fluorosilanes or chlorosilanes such as henicosyl-1,1,2,2-tetrahydrododecyldimethyltris(dimethylaminosilane), heptadecafluoro-1,1,2,2-tetrahydrodecyltrichlorosilane, nonafluoro-1,1,2,2-tetrahydrohexyltris(dimethylamino)silane, 3,3,3,4,4,5,5,6,6-nonafluorohexyltrichlorosilane, tridecafluoro-1,1,2,2-tetrahydrooctyltrichlorosilane, bis(tridecafluoro-1,1,2,2-tetrahydrooctyl)dimethylsiloxymethylchlorosilane, dodecyltrichlorosilane, dimethyldichlorosilane, or combinations thereof. Specific examples of membranes that can be used include PTFE SEP™ available from GVS SpA (Italy), GOTE-TEX® available from W.L. Gore and Associates, Inc. (USA), POREX® PTFE membranes available from Porex Filtration (USA), and FLUOROPORE™ available from MilliporeSigma (USA). In some examples, the polymer can have the form of a porous network, with pores that allow air or other gases to flow through the membrane. The polymer can be hydrophobic so that water is repelled from the hydrophobic pores. In some examples, the membrane can be impermeable to water. In other examples, the membrane can have a relatively high water entry pressure, meaning the pressure at which water will pass through the membrane. The water entry pressure of the membrane can be higher than the break pressure of the capillary break in the microfluidic structures described herein. In some examples, the water entry pressure of the membrane can be from 100 kPa to 300 kPa, or from 110 kPa to 250 kPa, or from 110 kPa, to 200 kPa. In certain examples, the membrane can have an average pore size from 0.2 μm to 0.5 μm.

In further examples, the gas-permeable liquid barrier can include a pore or pinhole having a hydrophobic surface. The hydrophobic surface can repel water and prevent water from flowing through the pore up to a pressure that is above the break pressure of the capillary break. However, gas can flow freely through the pore to allow the gas to escape from the sample chamber. In some examples, such a pore or pinhole can have a width or diameter from about 1 μm to about 5 μm, or from about 1 μm to about 2 μm.

Similarly, an additional capillary break can also be used as a gas-permeable liquid barrier. This additional capillary break can be designed similarly to the capillary break between the inlet microfluidic channel and the overflow chamber. However, this additional capillary break can have a higher break pressure than the capillary break between the inlet microfluidic channel and the overflow chamber. In some examples, the additional capillary break can have a narrow opening with a smaller width than the narrow opening of the first capillary break. In certain examples, the additional capillary break can have a narrow opening with a width from 1 μm to 15 μm or from 1 μm to 10 μm.

Other examples of gas-permeable liquid barriers can include labyrinth seals. Labyrinth seals can include a tortuous pathway that can slow or prevent the escape of liquid from the sample chamber. The tortuous pathway can have a small width or diameter and many turns or corners. Gas can flow more easily through the labyrinth seal. Thus, gas can flow out of the sample chamber through the labyrinth seal and then when the liquid in the sample chamber reaches the labyrinth seal, the flow of liquid can be stopped or slowed sufficiently that the liquid breaks the capillary break leading to the overflow chamber. After this, the liquid can preferentially flow into the overflow chamber rather than through the labyrinth seal.

Another example gas-permeable liquid barrier can include a material that swells or that otherwise becomes less permeable when contacted by water. For example, a dry hydrogel precursor material can be used. The dry hydrogel precursor can be in the form of a porous matrix or particles that include sufficient void space to allow gas to flow through the dry material. However, when a liquid such as water reaches the dry hydrogel precursor, the liquid can cause the dry hydrogel precursor to swell and convert to a hydrogel. The permeability of the hydrogel can be much lower, so that the hydrogel blocks further flow of liquid out of the sample chamber. Examples of dry hydrogel precursors can include sodium polyacrylate and other superabsorbent polymers.

As mentioned above, a capillary break can be positioned between the inlet microfluidic channel and the overflow chamber. The capillary break can have break pressure, below which liquid will not flow through the capillary break. Liquid can therefore preferentially flow into the sample chamber until the liquid fills the sample chamber and becomes constrained by the gas-permeable liquid barrier downstream of the sample chamber. Then, the pressure of the liquid can increase sufficiently to overcome the break pressure of the capillary break. Once the meniscus of the liquid passes through the capillary break and the liquid is present on both sides of the capillary break, the capillary break stops resisting the flow of liquid and the liquid can then freely flow into the overflow chamber.

The capillary break can be any structure that narrows the width of the microfluidic channel down to a narrow opening that has a smaller width than the inlet microfluidic channel, and which will prevent liquid from flowing through the narrow opening due to the capillary force or surface tension force of the liquid in the narrow opening. In some examples, the narrow opening can have a width from 2 μm to 20 μm. In other examples, the width can be from 2 μm to 15 μm or from 2 μm to 10 μm. Comparatively, the inlet microfluidic channel can have a larger width, such as from 30 μm to 1,000 μm or from 50 μm to 500 μm, for example. In further examples, the narrowed opening can have a width that is from 1% to 90% the width of the inlet microfluidic channel. In more specific examples, the narrowed opening can have a width that is from 2% to 60% or from 5% to 40% the width of the inlet microfluidic channel. Although the examples described herein include a single capillary break between the inlet microfluidic channel and the overflow chamber, in some examples there can be multiple capillary breaks in series. This can reduce the risk of the capillary break failing and allowing liquid to flow into the overflow chamber prematurely.

The overflow chamber can be positioned downstream of the capillary break so that liquid flows through the capillary break and into the overflow chamber. As mentioned above, the overflow chamber can be sized to accommodate any excess liquid volume that is expected to be in the liquid sample, which will not fit in the sample chamber. The overflow chamber can also have a sufficient volume so that the overflow chamber is partially filled by the excess liquid and some empty airspace remains in the overflow chamber. This can allow for additional liquid to flow into the overflow chamber during heating the sample liquid, due to thermal expansion of the liquid and/or expansion of bubbles that may be present in the sample chamber. In some examples, the overflow chamber can have an internal volume that is larger than the sample chamber. The specific volumes of the sample chamber and the overflow chamber can vary depending on the specific application of the microfluidic structure. In some examples, the sample chamber can have an internal volume from 5 μL to 200 μL, or from 10 μL to 100 μL, or from 10 μL to 50 μL. In further examples, the overflow chamber can have an internal volume from 5 μL to 400 μL, or from 10 μL to 200 μL, or from 20 μL to 100 μL.

A second gas-permeable liquid barrier can be connected downstream of the overflow chamber. This second gas-permeable liquid can allow gas to escape from the overflow chamber, just as the first gas-permeable liquid barrier allows gas to escape from the sample chamber. In various examples, any of the types of gas-permeable liquid barrier described above can be used for the second gas-permeable liquid barrier.

In further examples of the overfill-tolerant microfluidic structures described herein, the sample chamber may be divided into multiple chambers that can hold different fractions of a sample liquid. In some particular applications, a liquid sample that is introduced into the microfluidic structure may have a composition that varies in some way from one fraction of the sample to another. For example, in some cases the liquid that flows into the microfluidic structure first may be less valuable because the liquid is not well mixed at first, or because the first liquid introduced includes some sort of contaminant, or the liquid may have a different composition than the liquid flowing into the microfluidic structure later for some other reason. In such circumstances, it may not be desired to use the first fraction of liquid that is loaded into the microfluidic structure. Therefore, the sample chamber can be divided into a first fraction chamber and a second fraction chamber. The first fraction chamber can be downstream of the second fraction chamber. The first fraction of the sample liquid, which is of less interest, can flow into and fill the first fraction chamber. Then the second fraction chamber can be filled with liquid that is of more interest. In some examples, if heaters, sensors, or other equipment are used to process or measure the liquid, these components can be provided for the second fraction chamber but not the first fraction chamber. In additional examples, any number of fraction chambers can be included if multiple fractions of the sample liquid are desired. For example, the microfluidic structure can include a third fraction chamber, fourth fraction chamber, fifth fraction chamber, and so on.

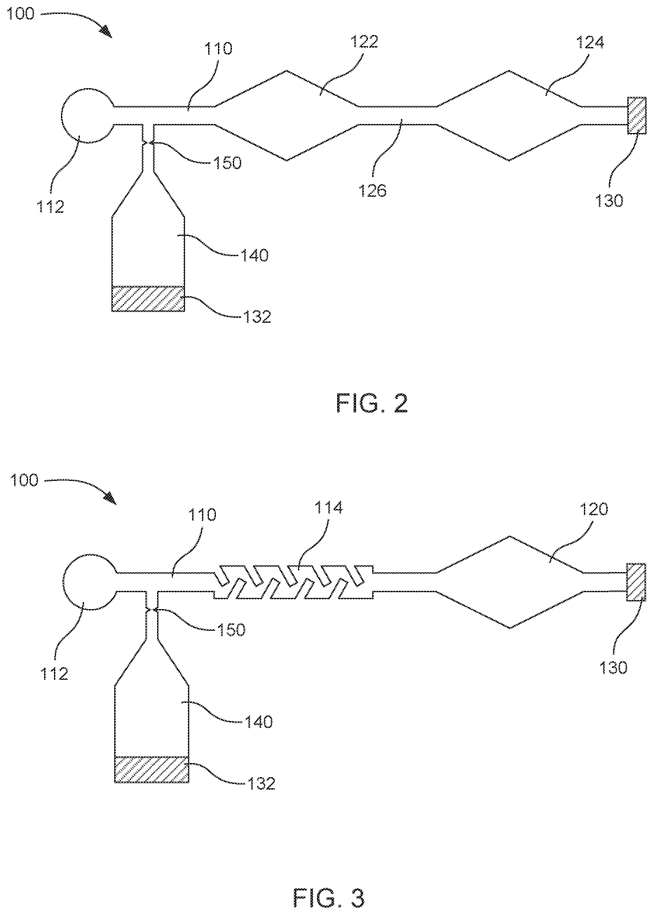

FIG. 2 shows one example overfill-tolerant microfluidic structure 100 that includes a first fraction chamber 122 and a second fraction chamber 124. A microfluidic connection channel 126 connects the second fraction chamber to the first fraction chamber. Together, the first fraction chamber and the second fraction chamber can make up a sample chamber. As in the previous examples, a gas-permeable liquid barrier 130 is positioned downstream of the first fraction chamber. An inlet microfluidic channel 110 allows liquid to flow from an inlet port 112 into the second fraction chamber, and then into the first fraction chamber. An overflow chamber 140 is connected to the inlet microfluidic channel upstream of the second fraction chamber. A capillary break is positioned between the inlet microfluidic channel and the overflow chamber. This example also includes a second gas-permeable liquid barrier 132 downstream of the overflow chamber as in the previous examples.

It can be useful to mix the sample liquid before the liquid fills the sample chamber in some cases. Some example microfluidic structures can include an in-line mixer in the inlet microfluidic channel, upstream of the sample chamber. In some examples, the in-line mixer can be upstream of the overflow chamber or downstream of the overflow chamber. The in-line mixer can be a passive mixer that mixes liquid in the inlet microfluidic channel using the flowing motion of the liquid. A variety of designs can be used for an in-line passive mixer. In some examples, the in-line mixer can include a microfluidic channel segment having internal baffles or ridges on the interior walls of the microfluidic channel. The baffles or ridges can cause the liquid to have a more turbulent flow, which can mix the liquid in the channel. Another design can include multiple sharp corners or turns in the microfluidic channel, which can also cause the flow to be more turbulent. Yet another design can include a microfluidic channel segment having a cross-section that contracts and expands. For example, the microfluidic channel can have a width that increases and decreases multiple times along the length of the microfluidic channel. In further examples, active mixers can also be used. Active mixers can include an actuator that mechanically mixes the liquid flowing through the inlet microfluidic channel.

FIG. 3 shows an example overfill-tolerant microfluidic structure 100 that includes a passive in-line mixer 114 in the inlet microfluidic channel 110. Liquid flows from the input port 112 through the mixer. This example mixer includes herringbone baffles within the microfluidic channel. These can make the liquid flow more turbulently and thereby mix the liquid as the liquid flows through the mixer. The mixer is positioned upstream of the sample chamber 120 so that the liquid in the sample chamber can be well-mixed. In this example, the mixer is downstream of the overflow chamber 140. This may be acceptable because there is no reason to mix the excess liquid that will go into the overflow chamber. As in the previous examples, this example also includes a gas-permeable liquid barrier 130 downstream of the sample chamber, a second gas-permeable liquid barrier 132 downstream of the overflow chamber, and a capillary break 150 between the inlet microfluidic channel and the overflow chamber.

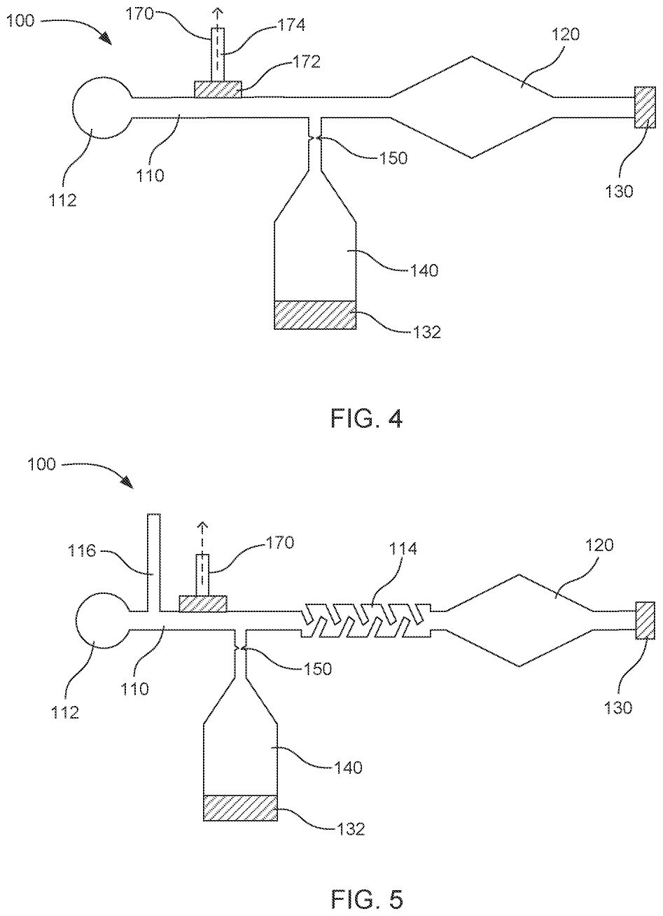

Another example overfill-tolerant microfluidic structure 100 is shown in FIG. 4. This example includes a bubble remover 170 on the inlet microfluidic channel 110. The bubble remover can remove gas bubbles from the liquid before the liquid flows into the sample chamber 120. In this example, the bubble remover is made up of a gas-permeable membrane 172 with a suction channel 174 that applies suction through the membrane. The gas-permeable membrane can be impermeable to liquid, so that gas bubbles are drawn through the membrane by the suction while liquid remains in the inlet microfluidic channel. The gas-permeable membrane can include any of the gas-permeable membrane materials described above. In this example, a sample liquid can flow from the input port 112 through the inlet microfluidic channel and past the bubble remover, where air bubbles can be removed from the liquid. The liquid can then flow into the sample chamber and fill the sample chamber with liquid that has a reduced air bubble content. As in previous examples, the liquid can fill the sample chamber and then the liquid can flow through the capillary break 150 into the overflow chamber 140. Again, a gas-permeable liquid barrier 130 can be connected downstream of the sample chamber, and a second gas-permeable liquid barrier 132 can be connected downstream of the overflow chamber.

In further examples, multiple inlet ports can be included so that multiple liquids can be introduced and mixed together in the microfluidic structure. An in-line mixer can be particularly useful in this type of example. In further examples, a combination of the features described herein can be used together in a single microfluidic structure. FIG. 5 shows one example overfill-tolerant microfluidic structure 100 that includes a combination of the above features. This example includes an inlet port 112 for loading fluid into an inlet microfluidic channel 110. A second inlet channel 116 connects to the inlet microfluidic channel so that a second liquid sample can be mixed with the first liquid sample. A bubble remover 170 is on the inlet microfluidic channel. The bubble remover can remove air bubbles from the liquid flowing past. In some examples, the bubble remover can include a gas-permeable membrane in contact with the liquid in the inlet microfluidic channel, and a suction channel on the opposite side of the gas-permeable membrane. The suction channel can apply suction through the gas-permeable membrane to draw air bubbles out of the liquid in the inlet microfluidic channel, while the gas-permeable membrane can prevent the liquid from escaping from the channel. The liquid then flows through an in-line mixer 114. The mixed liquid flows into the sample chamber 120 and fills the sample chamber until the liquid contacts the gas-permeable liquid barrier 130. After the sample chamber is full, the liquid can flow through the capillary break 150 and into the overflow chamber 140. Gas from the overflow chamber can escape through a second gas-permeable liquid barrier 132.

In some examples, the liquid in the sample chamber can be observed using a sensor such as an optical sensor. In certain examples, an optical sensor can be used to detect fluorescence or to make colorimetric measurements of the liquid. However, the presence of air bubbles in the sample chamber can interfere with such measurements. If an air bubble is present at the location where the sensor is to measure a property of the liquid, then the measurement can be incorrect because the sensor is measuring the air bubble instead of the liquid. To prevent this inaccuracy, the sample chamber can include bubble-excluding regions. These regions can be made by forming an area of increased hydrophilicity on an interior surface of the sample chamber. In other words, the bubble-excluding region can have a smaller contact angle with water compared to surrounding regions of the sample chamber. This can cause the liquid in the sample chamber to preferentially occupy the space in the bubble exclusion region, instead of an air bubble. Thus, if air bubbles are present in the sample chamber, the bubbles can be excluded from the bubble-excluding region. Sensors can be positioned to interrogate the liquid in the bubble-excluding region to ensure that the sensors are measuring the liquid and not the air bubbles. In some examples, the hydrophilicity of the bubble-excluding region can be increased by treating the surface with a plasma treatment. The plasma treatment can include oxygen plasma or a mixture of oxygen and nitrogen plasma to form a light oxidation layer on the surface. In other examples, the surface of the bubble-excluding region can be functionalized with hydrophilic silanes. Some example hydrophilic silanes include bis(3-cyanopropyl)dimethoxysilane, N-(2-N-benzylaminoethyl)-3-trimethoxysilylpropylammonium chloride, bis(3-trimethoxysilylpropyl)-N-methylamine, and others.

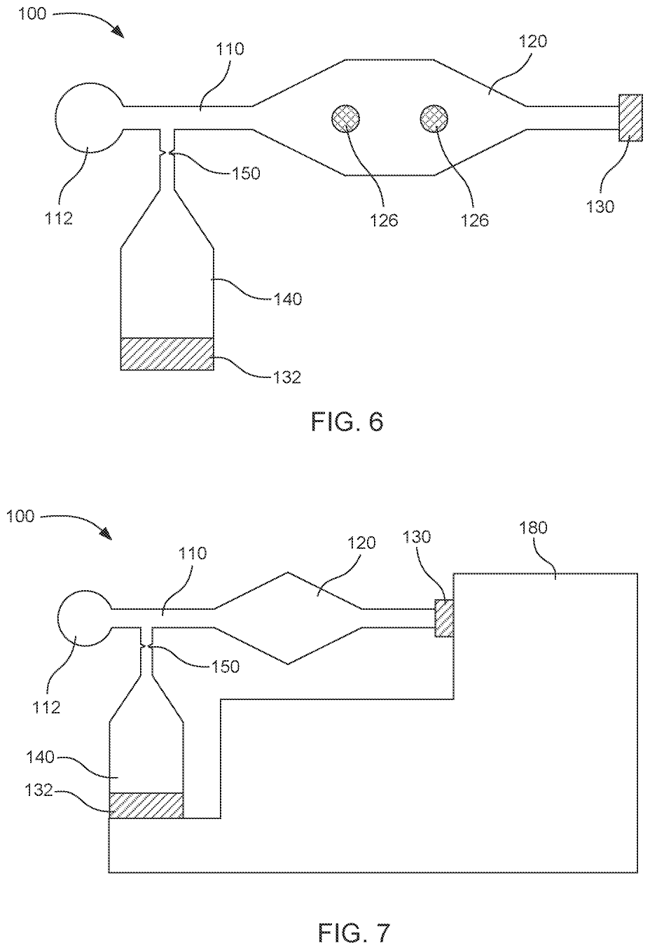

FIG. 6 shows one such example overfill-tolerant microfluidic structure 100. This example includes a sample chamber 120 that has two bubble excluding regions 126. The bubble-excluding regions can be made by, for example, applying a hydrophilic coating to the interior surface of the sample chamber in these regions. This example also includes similar components to other examples, such as an inlet port 112, an inlet microfluidic channel 110, a gas-permeable liquid barrier 130, a second gas permeable liquid barrier 132, an overflow chamber 140, and a capillary break 150.

Although the gas-permeable liquid barrier can normally prevent any liquid from escaping out of the sample chamber and the overflow chamber, it is possible that a small amount of liquid or other material from a sample may pass through the barrier in an aerosolized form. For example, the sample liquid may include a particular nucleic acid to be amplified. Nucleic acid amplification tests are often very sensitive, so that a very small amount nucleic acid in the wrong place on a test device could cause a false positive or other incorrect test result. Therefore, if a very small amount of aerosolized nucleic acid escapes through the gas-permeable liquid barrier, this could potentially cause incorrect test results. Accordingly, in some examples the overfill-tolerant microfluidic structure can include a containment chamber connected downstream of the gas-permeable liquid barrier. If any aerosolized material passes through the gas-permeable liquid barrier, then this aerosolized material can still be contained by the containment chamber. In some examples, the containment chamber can be a sealed chamber that is filled with air. In further examples, the containment chamber can have a significantly larger volume compared to the volume of the sample chamber. This can allow air in the sample chamber to be pushed into the containment chamber when the sample chamber is filled with liquid, without causing a large back pressure from the increase in the amount of air in the containment chamber. In still further examples, air that escapes from the overflow chamber through the second gas-permeable liquid barrier can also go into the containment chamber.

FIG. 7 shows an example overfill-tolerant microfluidic structure 100 that includes a containment chamber 180. The containment chamber is connected to the gas-permeable liquid barrier 130 and the second gas-permeable liquid barrier 132 downstream of the sample chamber 120 and the overflow chamber 140. This example also includes an input port 112, an inlet microfluidic channel 110, and a capillary break 150 as in the previous examples.

Although the previous examples shown in the figures have been depicted as two-dimensional designs in which the various microfluidic channels and chambers are in a single plane, other examples can have a variety of other designs in three dimensions. For example, the sample chamber or the overflow chamber can be positioned above or below a microfluidic channel. In certain examples, the overflow chamber can be positioned above or below the sample chamber. Thus, when viewed from above, the chambers and microfluidic channels can overlap one another.

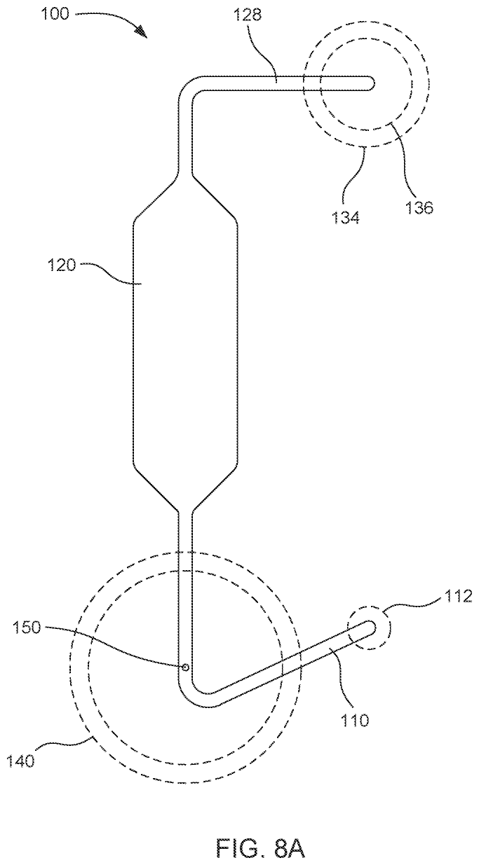

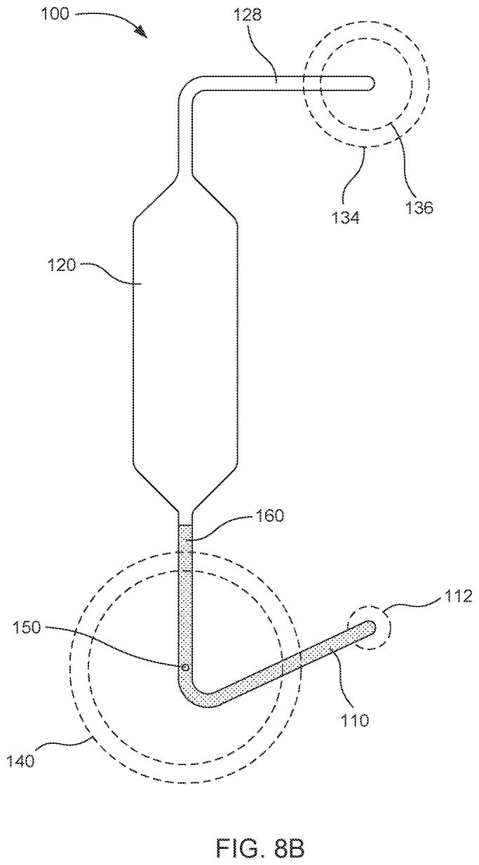

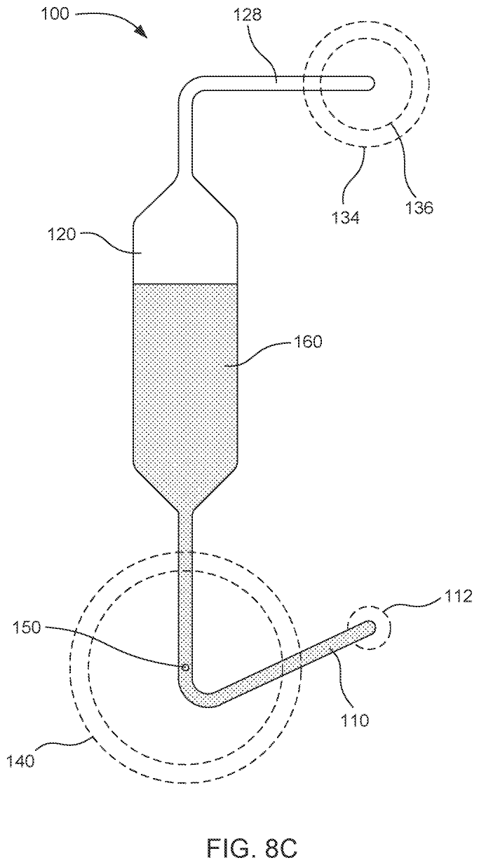

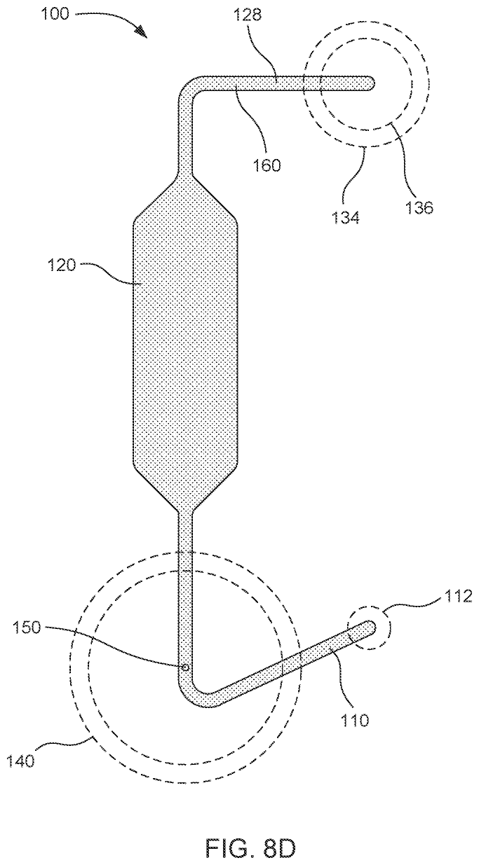

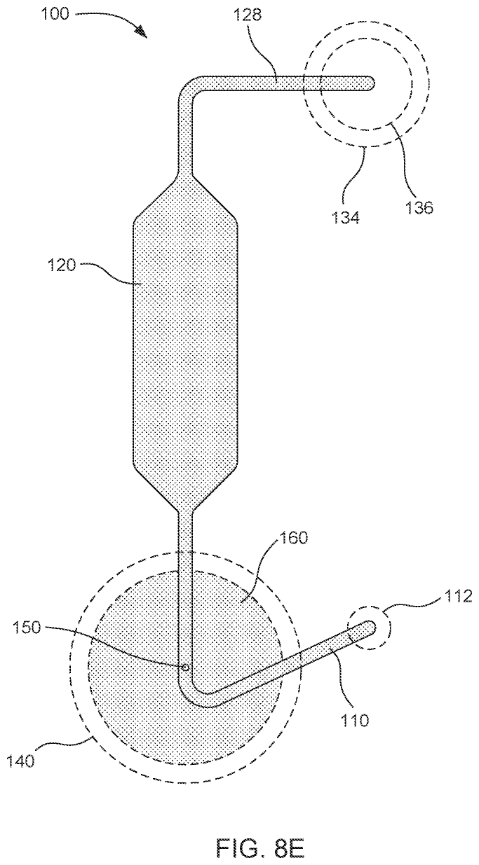

FIGS. 8A-8E show another example overfill-tolerant microfluidic structure 100. This figure shows a top-down schematic view of the structure. A sample chamber 120 and an inlet microfluidic channel 110 are shown in solid lines. A downstream microfluidic channel segment 128 is also included downstream of the sample chamber. The sample chamber and the microfluidic channels are all shown in solid lines because these components are together in one layer of the structure. Additionally, a capillary break 150 is formed as a small pinhole opening in the ceiling of the inlet microfluidic channel. The capillary break opening leads to an overflow chamber 140. The overflow chamber is shown in dashed lines because the overflow chamber is in a different layer, above the inlet microfluidic channel and the sample chamber. An inlet port 112 is also shown in dashed lines because the inlet port is in a different layer above the inlet microfluidic channel. The segment of microfluidic channel downstream from the sample chamber leads to an outlet 134 that can be plugged with a gas-permeable membrane disc 136. The outlet and the gas-permeable membrane disc are shown in dashed lines in this figure because they are also above the layer that includes the sample chamber and the microfluidic channels.

FIG. 8B shows how liquid 160 can be loaded into the inlet microfluidic channel 110. The liquid flows past the capillary break because the liquid does not have sufficient pressure to overcome the break pressure of the capillary break. FIG. 8C shows the liquid flowing into the sample chamber 120. In FIG. 8D, the liquid has completely filled the sample chamber and the liquid has flowed through the downstream microfluidic channel segment 128 and reached the gas-permeable membrane disc 136 at the outlet 134. FIG. 8E shows the liquid flowing into the overflow chamber 140 after the pressure of the liquid increases sufficiently to overcome the break pressure of the capillary break 150.

Accordingly, several examples of the overfill-tolerant microfluidic structures have been described. The overfill-tolerant microfluidic structures can include the various components in a single plane, or in multiple planes. The structures can include any of the features described above, such as microfluidic channels, sample chambers, overflow chambers, capillary breaks, gas-permeable liquid barriers, mixers, bubble removers, additional inlets, bubble excluding regions, sensors, heaters, containment chambers, and others. These features can be arranged in a three-dimensional space in any suitable arrangement. In many examples, the force of gravity may not be particularly significant in the operation of the microfluidic structures. Therefore, the microfluidic structures can be oriented in any direction.

### Microfluidic Devices

The present disclosure also describes microfluidic devices. In some examples, the microfluidic devices can include the overfill-tolerant microfluidic structures described above. The overfill-tolerant microfluidic structure can be formed on a substrate. In further examples, the substrate can also include a heater on or embedded in the substrate. Thus, the microfluidic device can be used for a process that involves heating a sample liquid.

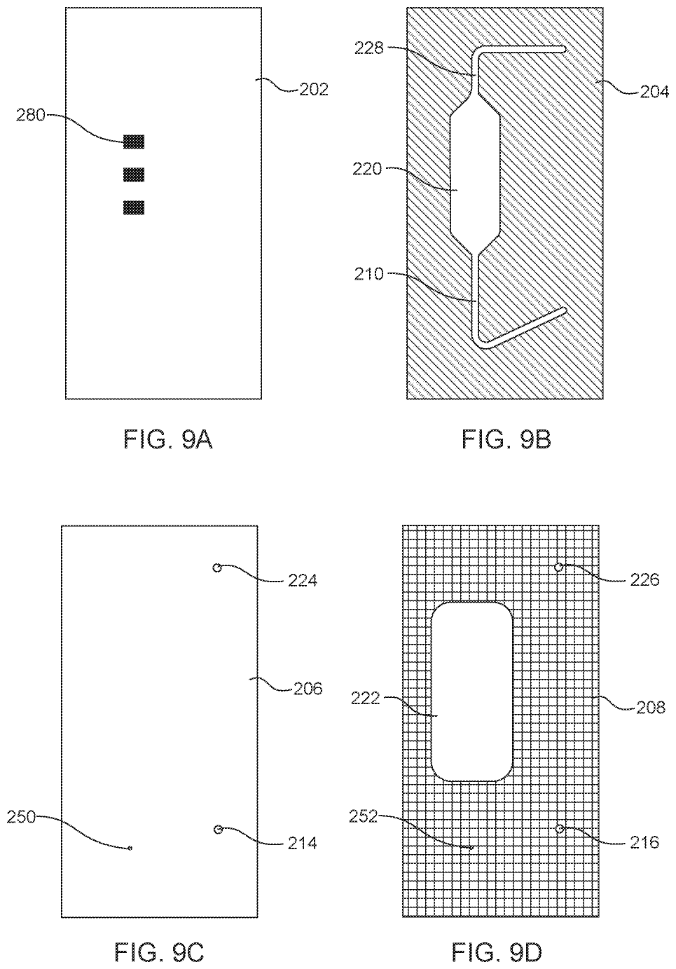

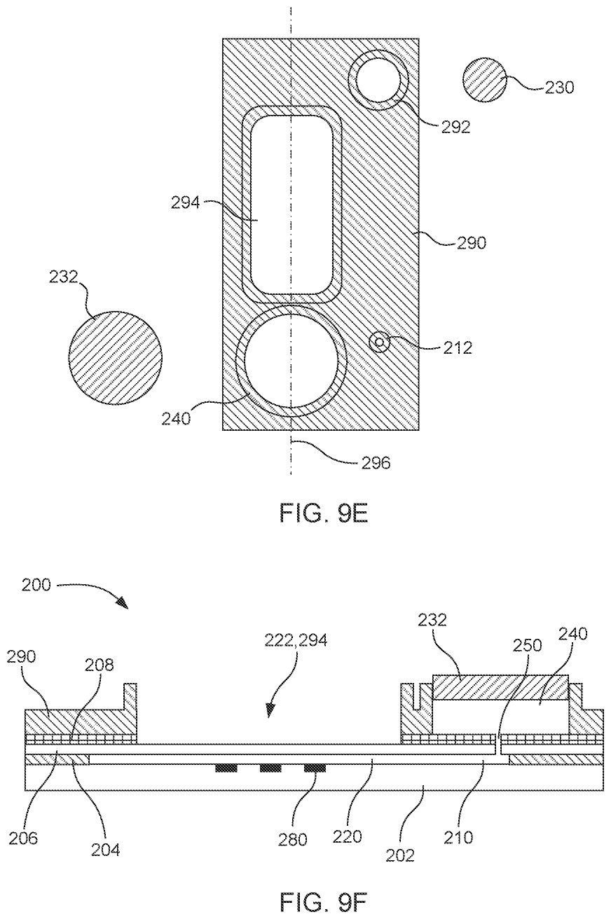

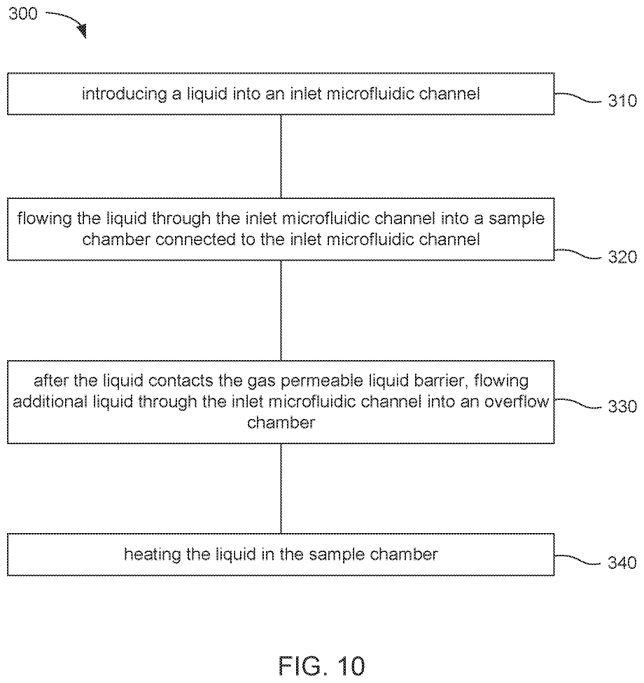

FIGS. 9A-9F show various views of an example microfluidic device 200 in accordance with the present disclosure. The microfluidic device is made up of several layers of material. FIGS. 9A-9E show individual layers of the microfluidic device, and FIG. 9F shows a side cross-sectional view of the assembled microfluidic device. FIG. 9A shows a substrate 202 that includes three thermal resistor heaters 280. The thermal resistor heaters can be formed as layers of electrically conductive material deposited on or embedded within the substrate. In further examples, the substrate can include electrically conductive traces (not shown) leading to the heaters to allow the heaters to be powered by a power source. The substrate can include a variety of materials, such as single crystalline silicon, polycrystalline silicon, gallium arsenide, glass, silica, ceramics, indium tin oxide, a semiconducting material, a printed circuit board, a polyimide film, plastic, metal sapphire, or a combination thereof. Some plastics that can be used include polycarbonate, cyclic olefin copolymer, acrylic, and others. Some metals that can be used include aluminum, copper, stainless steel, and others. In a particular example, the substrate can have a thickness from 500 μm to 5 mm, or from 500 μm to 2 mm, or from 500 μm to 1 mm.

FIG. 9B shows a microfluidic layer 204 that defines an inlet microfluidic channel 210, a sample chamber 220, and a downstream microfluidic channel segment 228 that is connected to the downstream end of the sample chamber. The microfluidic layer can be made of a variety of materials. In some examples, the microfluidic layer can be formed photolithographically using a photoresist. In one such example, the layer can be formed from an epoxy-based photoresist, such as SU-8 or SU-8 2000 photoresist, which are epoxy-based negative photoresists. Specifically, SU-8 and SU-8 200 are Bisphenol A Novolac epoxy-based photoresists that are available from various sources, including MicroChem Corp. These materials can be exposed to UV light to become crosslinked, while portions that are unexposed can remain soluble in a solvent and can be washed away to leave voids. In other examples, the microfluidic layer can be made from a high temperature pressure sensitive adhesive sheet, an ultra-violet curable adhesive, or another material. In some examples, the microfluidic layer can have a thickness from 2 μm to 1000 μm, or from 2 μm to 500 μm, or from 2 μm to 100 μm. The inlet microfluidic channel and the downstream microfluidic channel segment can be formed having a width from 2 μm to 1000 μm, from 10 μm to 500 μm, from 20 μm to about 100 μm, or from 40 μm to 100 μm, in some examples.

FIG. 9C shows a transparent film 206 that is placed over the top of the microfluidic layer. This transparent film layer forms a ceiling for the inlet microfluidic channel, the sample chamber, and the downstream microfluidic channel segment. The transparent film incudes a capillary break opening 250. This opening is positioned so that the opening will be in the ceiling of the inlet microfluidic channel when the device is assembled. The capillary break opening can have a width that is less than the width of the inlet microfluidic channel, as explained above. The transparent film layer also includes an inlet port opening 214 and an outlet opening 224. The inlet port opening lines up with the inlet microfluidic channel to allow liquid to flow from an inlet port into the inlet microfluidic channel. The outlet opening lines up with the end of the downstream microfluidic channel segment. When the sample chamber fills with liquid, air flows out of the sample chamber through the downstream microfluidic channel segment, and through the outlet opening. In some examples, the transparent film can be made of polycarbonate, cyclic olefin copolymer, a dry photoresist layer, or another material.

FIG. 9D shows an adhesive layer 208. The adhesive layer is positioned over the transparent film. The adhesive layer includes a capillary break opening 252, an inlet port opening 216, and an outlet pore opening 226. These openings line up with the corresponding openings on the transparent film layer beneath the adhesive layer. The adhesive layer also includes a sample chamber view window 222. The adhesive layer can be made from a pressure sensitive adhesive sheet, an ultra-violet curable adhesive, or another material.

FIG. 9E shows a top frame 290. The top frame can be a rigid frame that is placed over the top of the adhesive layer. In some examples, the top frame can be molded from a rigid material such as a plastic. Examples include polycarbonate, cyclic olefin copolymer, polystyrene, acrylonitrile butadiene styrene, or other plastics. The top frame can include an inlet port 212, an overflow chamber 240, and an outlet port 292. The inlet port can line up with the inlet openings of the lower layers and the inlet microfluidic channel of the microfluidic layer. In some examples, a liquid sample can be introduced into the inlet port using a syringe, pipette, hose, or other liquid source. The overflow chamber can be positioned over the capillary break openings in the layers below. When the break pressure of the capillary break is overcome, liquid can flow through the capillary break opening into the overflow chamber. The outlet port lines up with the outlet openings in the layers below and the downstream end of the downstream microfluidic channel segment. When air is displaced from the sample chamber, the air can flow out of the outlet port. FIG. 9E also shows a first gas-permeable membrane disc 230 and a second gas-permeable membrane disc 232. The first disc can fit into the top of the overflow chamber. A space can be left empty under the membrane disc so that there is sufficient volume inside the overflow chamber to accommodate excess liquid from a liquid sample. Similarly, the second membrane disc can fit into the outlet port. The second membrane disc can prevent liquid from flowing out of the outlet port, but air can be allowed to flow out through the membrane disc. The top frame also includes a sample chamber view window 294 that allows the sample chamber to be viewed by a user and/or by sensors such as optical sensors.

FIG. 9F shows a side cross-sectional view of the assembled microfluidic device 200, with the cross-section taken along a plane corresponding to dashed line 296 in FIG. 9E. The substrate 202 is positioned at the bottom of the microfluidic device. Three heaters 280 are embedded in the surface of the substrate. The sample chamber 220 is directly above the heaters. This can allow the heaters to heat the liquid in the sample chamber. The transparent film 206 forms the ceiling of the sample chamber. The adhesive layer 208 and the top frame 290 are positioned over the transparent film. As explained above, the adhesive layer and the top frame include a sample chamber view window 222,294 that allows the sample chamber to be viewed through the transparent film. The overflow chamber 240 is connected to the inlet microfluidic channel 210 by the capillary break 250. The second gas-permeable membrane disc 232 is placed over the overflow chamber in the top frame. The inlet port, outlet port and the first gas-permeable membrane disc are not shown in this figure because these components are not located on the dashed line 296.

In further examples, the microfluidic devices described herein can include microfluidic structures having any of the features and components described above. Some features can be formed as a part of the microfluidic layer. For example, a sample chamber can be divided into a first fraction chamber and a second chamber by forming such a pattern in the microfluidic layer. Additional inlet channels can be added to the microfluidic layer. An in-line mixer can be formed in the microfluidic layer by patterning the inlet microfluidic channel to have baffles or turns that mix the liquid. Other features can be formed as parts of other layers of the microfluidic device. In one example, a bubble remover can be added by including an additional gas-permeable membrane with a suction channel. These can be placed in the top frame, for example.

In certain examples, the microfluidic device can be in the form of a cartridge that can be used together with a sample processing system. For example, the cartridge can be designed to be a single-use disposable cartridge and the rest of the system can be reusable. In some examples, the cartridge can include a heater or heaters built into the substrate of the cartridge, as in the example microfluidic device described above. In other examples, the system can include reusable heaters that can be aligned with the cartridge when the cartridge is loaded in the system. The system can also include a sensor or sensors for measuring a property of a sample liquid in the cartridge. The system can also include additional components, such as a power source and an electronic controller. The power source can supply power to the heaters and sensors in the system, while the electronic controller can utilize the heaters and sensors to process a sample liquid.

### Methods of Heating a Liquid Sample

The present disclosure also describes methods of heating a liquid sample. These methods can be performed, in some examples, using the overfill-tolerant microfluidic structures and the microfluidic devices described above. FIG. is a flowchart illustrating one example method 300 of heating a liquid sample. This method includes: introducing a liquid into an inlet microfluidic channel 310 and flowing the liquid through the inlet microfluidic channel into a sample chamber connected to the inlet microfluidic channel 320. A gas permeable liquid barrier can be connected to the sample chamber and positioned to allow gas to flow out of the sample chamber as the gas is displaced by the liquid flowing into the sample chamber from the inlet microfluidic channel. The liquid can continue to flow into the sample chamber until the liquid contacts the gas permeable liquid barrier. The method also includes: after the liquid contacts the gas permeable liquid barrier, flowing additional liquid through the inlet microfluidic channel into an overflow chamber 330 and heating the liquid in the sample chamber 340. The overflow chamber can be connected to the inlet microfluidic channel upstream of the sample chamber, and a capillary break can be positioned between the inlet microfluidic channel and the overflow chamber. The capillary break can include a narrowed opening with a smaller width than a width of the inlet microfluidic channel. The capillary break can prevent liquid from passing up to a break pressure. The gas-permeable liquid barrier can allow gas to flow out of the sample chamber at a pressure lower than the break pressure, but can prevent liquid from flowing out of the sample chamber at the break pressure.

This example and similar methods can be useful when the total volume of the liquid sample is not precisely known. As explained above, the sample chamber can be designed to be smaller than the expected sample size, and the overflow chamber can be designed to have more than enough volume to accommodate the excess liquid of the sample. Any of the additional features of the microfluidic structures and microfluidic devices described above can also be used in the methods. For example, a method can include mixing the liquid using an in-line mixer in the inlet microfluidic channel upstream of the sample chamber. Another method can include containing any aerosolized material that passes through the gas-permeable liquid barriers using a containment chamber.

The methods and devices described herein can be used for a variety of microfluidic sample processing applications. In some specific examples, the methods can include amplifying target nucleic acids using a polymerase chain reaction (PCR) process. PCR is a process that can rapidly copy millions to billions of copies of a very small nucleic acid sample, such as DNA or RNA. In the PCR process, nucleic acid monomers can react to form many copies of the target nucleic acid. Therefore, if a single copy of the target nucleic acid was originally in the liquid sample, then the PCR process can create many more copies of that target nucleic acid in the sample chamber. The PCR process can involve thermal cycling, in which the temperature of the sample liquid is repeatedly raised and lowered. The heating can be accomplished using the heaters of the microfluidic devices described herein. The heaters can heat the sample liquid, and then the sample liquid can be allowed to cool to cycle the temperature. In some examples, thermal cycling can be performed between a low temperature and a high temperature that are both from 35° C. to 90° C., or from 35° C. to 60° C., or from 40° C. to 50° C.

The reagents involved in the PCR reaction can include the target nucleic acid, nucleic acid monomers, fluorescent probes that increase in fluorescence when they intercalate nucleic acids, primers, among others. In some examples, multiple reactants can be provided as a PCR master mix. PCR master mix reagents can include a mixture of multiple compounds that are used in a PCR process. These compounds can include DNA polymerase, nucleoside triphosphate, deoxyribose nucleoside triphosphate, magnesium chloride, magnesium sulfate, template DNA, forward primer, reverse primer, tris hydrochloride, potassium chloride, and others. Examples of commercially available PCR master mixes include TITANIUM TAQ ECODRYm premix, ADVANTAGE 2 ECODRYm premix (available from Takara Bio, Inc. Japan); Lyophilized Ready-to-Use and Load PCR Master Mix (available from Kerafast, Inc., USA); MAXIMO™ Dry-Master Mix (available from GenEon Technologies, USA), and others. In some examples, a sample liquid containing a nucleic acid can be introduced into the microfluidic device through an inlet port, and a separate master mix inlet can be used to introduce the master mix reagents.

In further examples, primers can be introduced separately from the other reagents. In some examples, primers can be introduced through another inlet port. In other examples, primers can be immobilized on an interior surface of the sample chamber. Primers are short single-stranded nucleic acids used in nucleic acid synthesis. In PCR processes, a pair of custom primers can be used to direct elongation of a nucleic acid being formed from opposite ends of the specific nucleic acid sequence that is being amplified. The primers can code for specific sites at either end of the sequence that is being amplified. Thus, specific pairs of primers can be selected to amplify a specific target nucleic acid sequence. In certain examples, the microfluidic device can be pre-treated with primers by introducing a solution of the desired primers into the sample chamber. The primers can then be lyophilized or immobilized on the interior surfaces of the sample chamber. Thus, the primers can be included in a dry state within the sample chamber at the time of manufacture. In certain examples, the primers can be immobilized on an interior surface of the sample chamber using linker molecules. The linker molecule can be a thermally labile linker, meaning that the linker can degrade or release the primer molecules at a certain temperature. Some examples of thermally labile linkers can include esters; sulfur-containing linkages such as disulfides, sulfonate, 5-membered cyclic dithiocarbonates, trithiocarbonates, or sulfites; nitrogen-containing linkages such as acylhydrazones, alkoxyamines, azlactones, Schiff base, hindered ureas, aminals, or carbamates; orthoesters, carbonates, acetals, hemiacetals, olefinic bonds, vicinal tricarbonyls, peroxide bonds, and others. Thus, the primer molecules can remain immobilized until heat is applied, such as when the system is heated up to an appropriate temperature for a nucleic acid amplification process.

It is to be understood that this disclosure is not limited to the particular processes and materials disclosed herein because such processes and materials may vary somewhat. It is also to be understood that the terminology used herein is used for the purpose of describing particular examples. The terms are not intended to be limiting because the scope of the present disclosure is intended to be limited by the appended claims and equivalents thereof.

It is noted that, as used in this specification and the appended claims, the singular forms “a,” “an,” and “the” include plural referents unless the context clearly dictates otherwise.

As used herein, the term “substantial” or “substantially” when used in reference to a quantity or amount of a material, or a specific characteristic thereof, refers to an amount that is sufficient to provide an effect that the material or characteristic was intended to provide. The exact degree of deviation allowable may in some cases depend on the specific context.

As used herein, the term “about” is used to provide flexibility to a numerical range endpoint by providing that a given value may be “a little above” or “a little below” the endpoint. The degree of flexibility of this term can be dictated by the particular variable and determined based on the associated description herein.

As used herein, a plurality of items, structural elements, compositional elements, and/or materials may be presented in a common list for convenience. However, these lists should be construed as though the members of the list are individually identified as a separate and unique members. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include the numerical values explicitly recited as the limits of the range, and also to include individual numerical values or sub-ranges encompassed within that range as if the numerical values and sub-ranges are explicitly recited. As an illustration, a numerical range of “about 1 wt % to about 5 wt %” should be interpreted to include the explicitly recited values of about 1 wt % to about 5 wt %, and also include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting a single numerical value. Furthermore, such an interpretation should apply regardless of the breadth of the range or the characteristics being described.

### EXAMPLE

The following illustrates an example of the present disclosure. However, it is to be understood that the following are merely illustrative of the application of the principles of the present disclosure. Numerous modifications and alternative compositions, methods, and systems may be devised without departing from the scope of the present disclosure.

### Example 1—Microfluidic Device

A microfluidic device was assembled having a design similar to the design shown in FIGS. 9A-9F. A printed circuit board was used as a substrate. A microfluidic layer was adhered to the substrate. The microfluidic layer defined an inlet microfluidic channel, a sample chamber, and a downstream microfluidic channel leading to an air outlet. A transparent polycarbonate film was then laid over the microfluidic layer, and a molded plastic top frame was adhered using a layer of pressure sensitive adhesive. A capillary break opening was formed in the transparent film, pressure sensitive adhesive layer, and the top frame. An air outlet was also formed in the transparent film, the pressure sensitive adhesive layer, and the top frame. An inlet port opening was also formed in the transparent film, the pressure sensitive adhesive layer, and the top frame.

A sample liquid (blue dyed water) was injected into the microfluidic device through the inlet port to test filling of the sample chamber. Multiple sample chambers were assembled and tested in this way. The sample chambers successfully filled with the water, and excess water flowed through the capillary break to the overflow chambers. It was found that some of the sample chambers contained small air bubbles after filling.

Another series of microfluidic devices was assembled having the same design, except a layer of KAPTON® tape (from E.I. du Pont de Nemours and Company, USA) was applied to the printed circuit board on the floor of the sample chamber. The experiment was repeated by filling the sample chambers with blue dyed water. It was found that the KAPTON® tape slightly reduced the formation of air bubbles in the sample chambers.

While the present technology has been described with reference to certain examples, various modifications, changes, omissions, and substitutions can be made without departing from the disclosure.

1. An overfill-tolerant microfluidic structure comprising:
an inlet microfluidic channel;
a sample chamber connected to the inlet microfluidic channel to receive liquid from the inlet microfluidic channel;
a gas-permeable liquid barrier connected to the sample chamber and positioned to allow gas to flow out of the sample chamber;
an overflow chamber connected to the inlet microfluidic channel; and
a capillary break positioned between the inlet microfluidic channel and the overflow chamber, wherein the capillary break comprises a narrowed opening with a smaller width than a width of the inlet microfluidic channel.

2. The overfill-tolerant microfluidic structure of claim 1, wherein the overflow chamber is connected upstream of the sample chamber.

3. The overfill-tolerant microfluidic structure of claim 1, wherein the capillary break prevents liquid from passing up to a break pressure and wherein the gas-permeable liquid barrier allows gas to flow out of the sample chamber at a pressure lower than the break pressure, but prevents liquid from flowing out of the sample chamber at the break pressure.

4. The overfill-tolerant microfluidic structure of claim 1, wherein the gas-permeable liquid barrier comprises a gas-permeable liquid-impermeable membrane, a pore having a hydrophobic surface, a labyrinth seal, a dry hydrogel precursor, or a second capillary break having a smaller width than the capillary break between the inlet microfluidic channel and the overflow chamber.

5. The overfill-tolerant microfluidic structure of claim 1, wherein the narrowed opening has a width from 2 μm to 20 μm.

6. The overfill-tolerant microfluidic structure of claim 1, further comprising a second gas-permeable liquid barrier connected to the overflow chamber and positioned to allow gas to flow out of the overflow chamber.

7. The overfill-tolerant microfluidic structure of claim 1, wherein the sample chamber comprises a first fraction chamber, a second fraction chamber upstream of the first fraction chamber, and a microfluidic connection channel connecting the first fraction chamber to the second fraction chamber.

8. The overfill-tolerant microfluidic structure of claim 1, further comprising a containment chamber connected downstream of the gas-permeable liquid barrier to contain aerosolized material that passes through the gas-permeable liquid barrier.

9. The overfill-tolerant microfluidic structure of claim 1, wherein the sample chamber comprises a bubble-excluding region having an area of increased hydrophilicity on an interior surface of the sample chamber compared to surrounding areas of the interior surface.

10. The overfill-tolerant microfluidic structure of claim 1, wherein the inlet microfluidic channel comprises an in-line mixer.

11. The overfill-tolerant microfluidic structure of claim 1, further comprising a bubble remover on the inlet microfluidic channel to remove gas bubbles from the liquid before the liquid flows into the sample chamber.

12. A microfluidic device comprising:
a substrate;
a heater on or embedded in the substrate; and
a microfluidic structure on the substrate, wherein the microfluidic structure comprises:
an inlet microfluidic channel,
a sample chamber connected to the inlet microfluidic channel to receive liquid from the inlet microfluidic channel, wherein the sample chamber is proximate to the heater,
a gas-permeable liquid barrier connected to the sample chamber and positioned to allow gas to flow out of the sample chamber,
an overflow chamber connected to the inlet microfluidic channel, and
a capillary break positioned between the inlet microfluidic channel and the overflow chamber, wherein the capillary break comprises a narrowed opening with a smaller width than a width of the inlet microfluidic channel.

13. The microfluidic device of claim 10, wherein the substrate comprises glass, silicon, a printed circuit board, a polyimide film, plastic, metal, sapphire, or a combination thereof.

14. A method of heating a liquid sample comprising:
introducing a liquid into an inlet microfluidic channel;
flowing the liquid through the inlet microfluidic channel into a sample chamber connected to the inlet microfluidic channel, wherein a gas permeable liquid barrier is connected to the sample chamber and positioned to allow gas to flow out of the sample chamber as the gas is displaced by the liquid flowing into the sample chamber from the inlet microfluidic channel, and wherein flowing the liquid into the sample chamber continues until the liquid contacts the gas permeable liquid barrier;
after the liquid contacts the gas permeable liquid barrier, flowing additional liquid through the inlet microfluidic channel into an overflow chamber that is connected to the inlet microfluidic channel, wherein a capillary break is positioned between the inlet microfluidic channel and the overflow chamber, wherein the capillary break comprises a narrowed opening with a smaller width than a width of the inlet microfluidic channel; and
heating the liquid in the sample chamber.

15. The method of claim 13, wherein the liquid comprises a target nucleic acid and a master mix reagent to amplify the target nucleic acid, and wherein the heating is repeated such that the target nucleic acid is amplified via a polymerase chain reaction process.
